(19) United States
(12) Patent Application Publication
(10) Pub. No.: US 2026/0199735 A1
(43) Pub. Date: Jul. 16, 2026

(54) CONNECTED STRENGTH SYSTEM FOR FITNESS EQUIPMENT

(71) Applicant: 4IIII INNOVATIONS INC., Cochrane, Alberta (CA)

(72) Inventors: Michael Donald MERCER, Cochrane, Alberta (CA); Jacob Kenneth PAWLUK, Cochrane, Alberta (CA); Dilan Peter Joseph TUFF-OVERES, Cochrane, Alberta (CA); Janelle CHUBEY, Cochrane, Alberta (CA); Tommy Wing Hong HUI, Cochrane, Alberta (CA)

(21) Appl. No.: 19/139,149

(22) PCT Filed: Dec. 15, 2023
(86) PCT No.: PCT/IB2023/000778

§ 371 Date: Jun. 13, 2025

Related U.S. Application Data
(60) Provisional application No. 63/433,259, filed on Dec. 16, 2022.

(51) Int. Cl. A63B 24/00; A63B 21/00; A63B 21/062; A63B 71/06; G16H 40/67

(52) U.S. Cl. CPC A63B 24/0062; A63B 21/0628; A63B 21/154; A63B 71/0622; G16H 40/67; A63B 2220/51; A63B 2220/58; A63B 2220/805; A63B 2220/833; A63B 2225/20; A63B 2225/50

(57) ABSTRACT

A connected system includes a sensor module with a sheave pin load cell sized and shaped to support a pulley of strength fitness equipment. The sensor module also include a sensor for sensing movement of the pulley as the pulley rotates. The sensor module includes a processor and memory storing machine-readable instructions that when executed by the processor cause the sensor module to: capture raw force data from the sheave pin load cell, capture raw movement data from the sensor, determine corrected force data from the raw force data based on calibration data, determine corrected movement data from the raw movement data based on the calibration data; and output the corrected force data and the corrected movement data via a communication interface. An interface module may receive and display the corrected force data and the corrected movement data.

### RELATED APPLICATION

This application claims priority to U.S. Patent Application No. 63/433,259, titled “Connected Strength System for Fitness Equipment Retrofit,” filed Dec. 16, 2022, and incorporated herein by reference in its entirety.

### BACKGROUND

Certain fitness equipment does not include sensory and electrical equipment capable of tracking training. Typical add-on devices removably clip onto a belt or cable of the machine (e.g., near a weight stack) to track movement and force. However, such devices are easily removed (stolen) or damaged by the activity of the machine. Further, these devices display data such as a repetition count, but do not provide a simple or generic way to digitally export or collect the exercise data, since they require the use of a proprietary wrist strap to connect with an app that is specific to a manufacturer of the device.

### SUMMARY

The present embodiments include the realization that a cable or belt of strength fitness equipment typically couples to a weight stack, and therefore any modification of the cable or belt that changes its length, such as to incorporate sensors for measuring force and/or repetitions for example, is undesirable. Further, it is realized that any device that attaches easily, also detaches easily and may therefore be easily lost (e.g., stolen or knocked off). The present embodiments solve these problems by providing a connected system that includes sensors that are quickly retrofitted to a pulley of the fitness equipment to measure force on, and movement of, the cable or belt. The sensors communicate with a computer module that processes the sensor data to calculate work (effort) performed by a user of the strength fitness equipment. For example, the sensors may measure force applied to the pulley and rotation of the pulley to determine one or more of displacement, speed, and repetitions of the exercise. Advantageously, the modification to the strength fitness equipment does not alter the length of the cable or belt and therefor does not disrupt operation of the machine. Advantageously, the connected system connects wirelessly with other devices (e.g., smart watches, smartphones, and other mobile devices) without requiring proprietary accessories.

In certain embodiments, the techniques described herein relate to a connected system for fitness equipment, including a sensor module having: a sheave pin load cell sized and shaped to support a pulley of the fitness equipment; a sensor for sensing movement of the pulley as the pulley rotates; a communication interface; a processor; and memory storing machine-readable instructions that when executed by the processor cause the sensor module to: capture raw force data from the sheave pin load cell; capture raw movement data from the sensor; determine corrected force data and from the raw force data based on calibration data; determine corrected movement data from the raw movement data based on the calibration data; and output the corrected force data and the corrected movement data via the communication interface.

In certain embodiments, the techniques described herein relate to a method for measuring performance of a user performing an exercise on fitness equipment, including: capturing, within a sensor module, raw force data from a sheave pin load cell supporting a pulley of the fitness equipment; capturing raw movement data defining movement of the pulley using a sensor and a pattern on the pulley; determining corrected force data from the raw force data based on calibration data; determining corrected movement data from the raw movement data based on the calibration data; and sending the corrected force data and the corrected movement data to an interface module.

In certain embodiments, the techniques described herein relate to a method for adding a connected system to fitness equipment, including: removing a sheave pin of a pulley carrying a cable or belt of the fitness machine; adding an optical pattern to at least one side of the pulley; mounting the pulley within the fitness machine using a sheave pin load cell, where the sheave pin load cell replaces the sheave pin; and calibrating the connected system by performing a predefined procedure on the fitness machine while the connected system is in a calibration mode that measures a force on the sheave pin load cell and measures movement of the pulley.

### BRIEF DESCRIPTION OF THE FIGURES

FIG. 1 is a schematic diagram illustrating one example prior art indoor strength fitness equipment that includes a supporting frame and a weight stack with selectable weights.

FIG. 2 is a schematic diagram illustrating one example improved indoor strength fitness equipment that includes a sensor module and an interface module for detecting user performance, in embodiments.

FIG. 3 is a schematic diagram illustrating a pulley, part of a frame, and part of a cable of indoor strength fitness equipment of FIG. 2 in further detail, and prior to fitting of sensor module, in embodiments.

FIGS. 4A and 4B are isometric views showing fitting of the sensor module to a pulley of the indoor strength fitness equipment of FIGS. 2 and 3, in embodiments.

FIG. 5 is a block diagram showing the sensor module of FIG. 2 in further example detail, in embodiments.

FIG. 6 is a block diagram showing the interface module of FIG. 2 in further example detail, in embodiments.

FIG. 7 is a block diagram illustrating example dataflow for the improved indoor strength fitness equipment of FIG. 2, in embodiments.

FIG. 8 is a block diagram illustrating one example state machine implemented within the sensor module of the indoor strength fitness equipment of FIG. 2, in embodiments.

FIG. 9 is a flowchart illustrating one example method for measuring performance of a user performing an exercise on fitness equipment, in embodiments.

FIG. 10 is a flowchart illustrating one example method for adding a connected system to a strength fitness machine, in embodiments.

### DETAILED DESCRIPTION OF THE EMBODIMENTS

The following embodiments and examples describe indoor strength fitness equipment. However, other types of fitness equipment may use the embodiments described herein without departing from the scope hereof. The types of fitness equipment may include a rowing machine. Further, the embodiments and example discuss the use of an optical pattern being sensed by optical sensors to measure rotational movement of a pulley; however, other types of sensors and patterns may be used without departing from the scope hereof. For example, magnetic sensors may detects a pattern of teeth on a pulley.

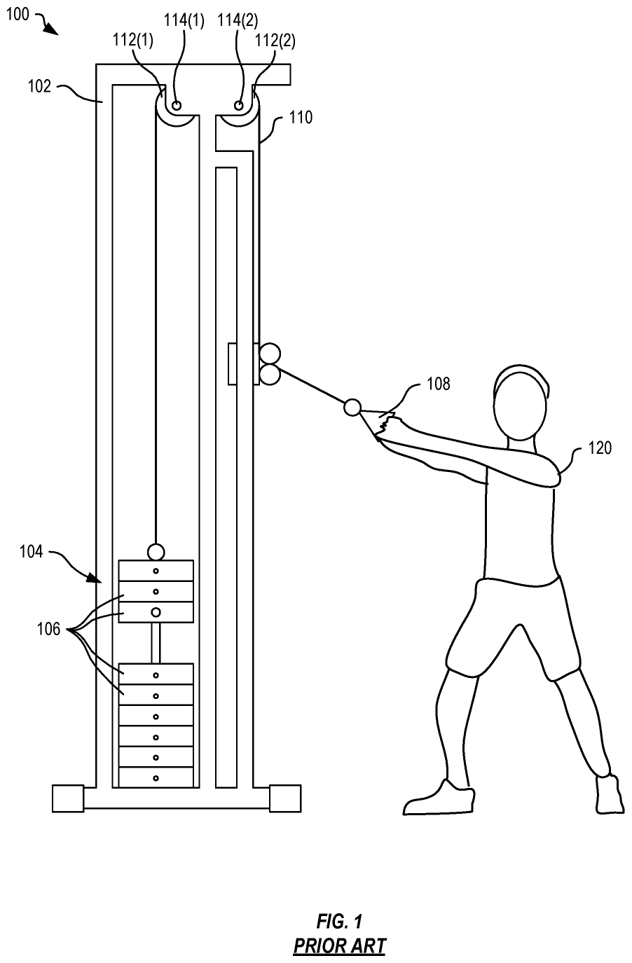

FIG. 1 is a schematic diagram illustrating prior art indoor strength fitness equipment 100 that includes a supporting frame 102, a weight stack 104 with selectable weights 106. A handle 108 couples with selected weights 106 via a cable 110 that passes over at least one pulley 112, wherein pulling on handle 108 applies a force to cable 110 to lift selectable weights 106. Indoor strength fitness equipment 100 is not smart and does not include sensors for sensing or tracking performance of a user 120. Accordingly, user 120 receives no indication of work performed when using indoor strength fitness equipment 100.

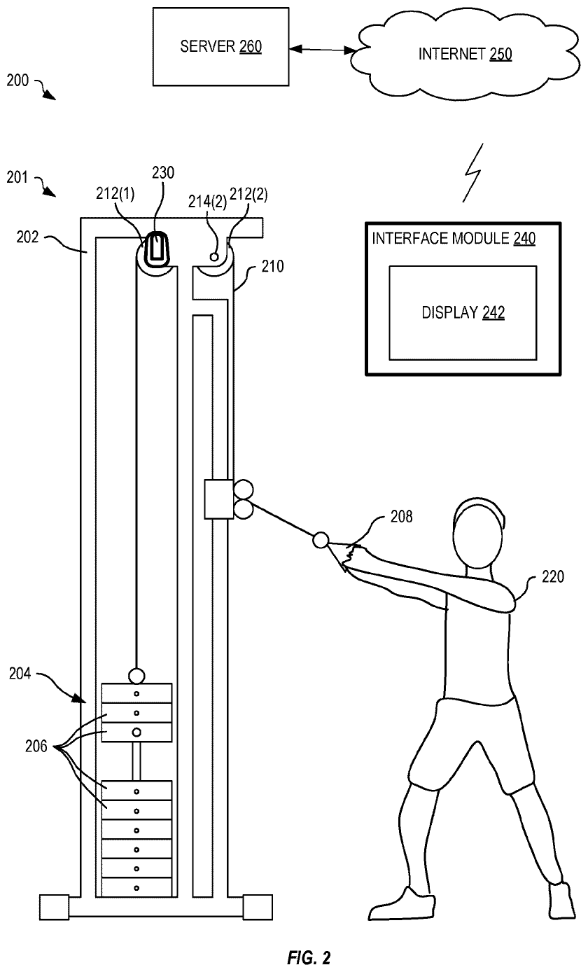

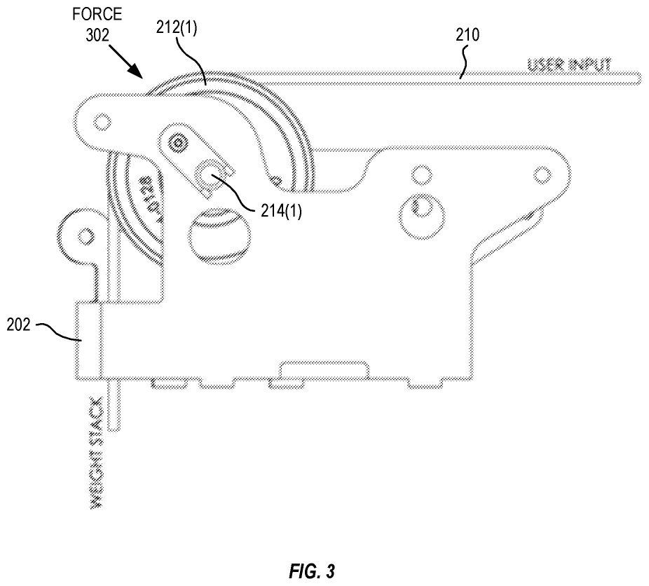

FIG. 2 is a schematic diagram illustrating one example connected system 200 fitted to an indoor strength fitness equipment 201. FIG. 3 is a schematic diagram illustrating pulley 212(1), part of frame 202, and part of cable 210 of indoor strength fitness equipment 201 of FIG. 2 in further detail, and prior to fitting of sensor module 230. FIGS. 2 and 3 are best viewed together with the following description.

Connected system 200 may be retrofitted to indoor strength fitness equipment 100 of FIG. 1, or may be included during manufacture of indoor strength fitness equipment 201. Connected system 200 includes a sensor module 230 that is configured with indoor strength fitness equipment 201 and an interface module 240 that may be mounted on indoor strength fitness equipment 201 or mounted separately therefrom.

Indoor strength fitness equipment 201 includes a supporting frame 202, a weight stack 204 with selectable weights 206. A handle 208 couples with the selected weights via a cable 210 that passes over at least one pulley 212(1) and 212(2) mounted on sheave pins 214(1) and 214(2), wherein pulling on handle 208 applies a force to cable 210 to lift selectable weights 206. As a user 220 pulls on handle 208, cable 210 applies a force 302 against pulley 212(1), which in turn applies force 302 to sheave pin 214(1) of pulley 212(1).

Connected system 200 (e.g., sensor module 230 and interface module 240) safely and accurately measures, records, and monitors a performance of user 220 using indoor strength fitness equipment 201. Particularly, sensor module 230 and interface module 240 advances functionality of indoor strength fitness equipment 201 to digitally connect user 220 with their data, as has become mainstream and expected in almost all areas of cardio and performance training. However, to date there are no effective retrofittable options to advance indoor strength fitness equipment.

Sensor module 230 and interface module 240 may have at least one of the following capabilities:
Measure load/weight
   with a weight accuracy of ±0.5 kg.
   with a weight repeatability of 99%.
Weight stack position
   Repetition counting based on position and load with an accuracy of 100%.
   Tracking of repetition tempo with an accuracy of 96%.
   Range of motion position with an accuracy of within 2 cm.
   Velocity of weight stack with an accuracy of 96%
User information
   Settings of fitness equipment that user used previously
   Previous weight value used for previous exercise sessions
   Historical data of users progress, weights, reps, sets
   Comparison between current progress and goals set by the user

Connected system 200 may also include the following capabilities: measure and display lift velocity, distance, and power, integrate with an Athlete Management System (AMS), include a web portal dashboard, be compatible with various smartphone devices (e.g., iOS and Android), and include a team setting as appropriate.

Advantageously, sensor module 230 and interface module 240 are easily retrofitted to existing indoor strength fitness equipment and are particularly suited to any cable or belt pull type strength fitness equipment. Connected system 200 may be retrofitted to ninety percent of existing cable/belt fitness equipment (e.g., indoor strength fitness equipment 100 of FIG. 1) in under ten minutes, for example.

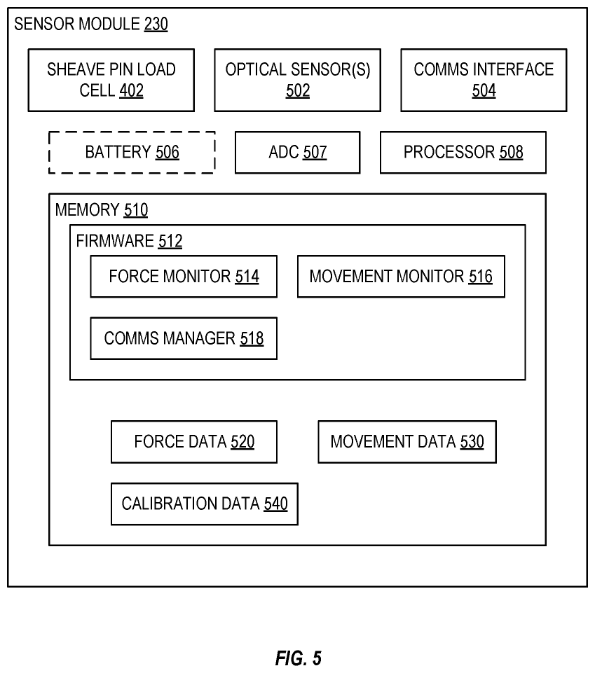

Interface module 240 may include a display 242 (e.g., a custom TFT screen) for displaying data collected and/or determined for exercises performed by user 220 on indoor strength fitness equipment 201. Interface module 240 may communicate, via the Internet 250 for example, with a server 260 (e.g., a remote server or cloud based service) that collects, stores, processes, and/or shares data captured by sensor module 230 and interface module 240. Interface module 240 may include a mount that attaches interface module 240 to frame 202 of indoor strength fitness equipment 201. Alternatively, interface module 240 may stand or attach to other structure independent of indoor strength fitness equipment 201. In certain embodiments, sensor module 230 and interface module 240 may communicate wirelessly, and sensor module 230 includes an independent power source (e.g., a battery-see optional battery 506, FIG. 5). In other embodiments, sensor module 230 and interface module 240 may be communicatively wired together where the wires also carry power from interface module 240 to sensor module 230, which accordingly does not require the battery. In certain embodiments, interface module 240 is implemented by an app running on a user's smartphone or other mobile device (e.g., smart watch).

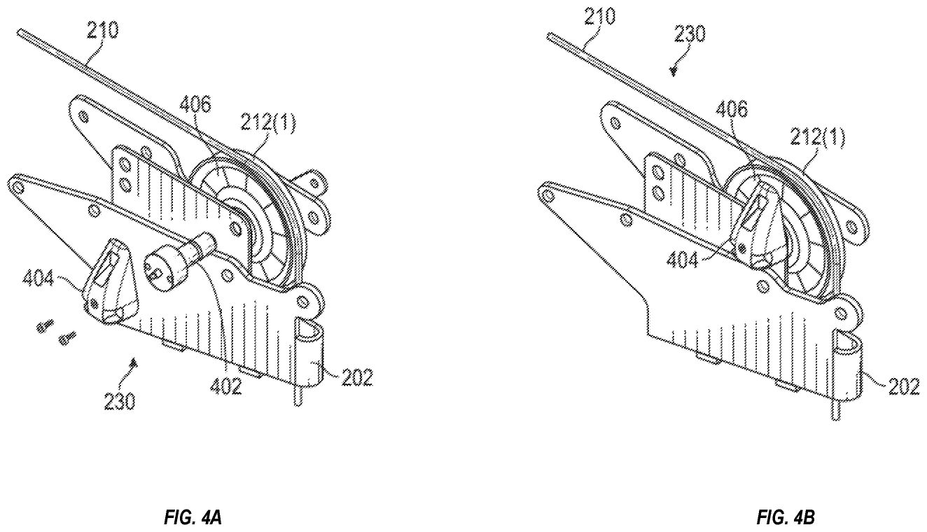

FIGS. 4A and 4B are perspective views showing sensor module 230 fitted to pulley 212(1) of indoor strength fitness equipment 201 of FIGS. 2 and 3. FIGS. 2, 3, 4A and 4B are best viewed together with the following description.

A sheave pin load cell 402 supports pulley 212(1) within frame 202 of indoor strength fitness equipment 201. For example, sheave pin 214(1) of pulley 212(1) is removed and replaced by sheave pin load cell 402, where sheave pin load cell 402 is sized and shaped to functionally support pulley 212(1) within frame 202. Sheave pin load cell 402 senses a force applied by pulley 212(1) to sheave pin load cell 402 as user 220 pulls on handle 208 causing cable 210 to lift selectable weights 206 of weight stack 204. A housing 404 of sensor module 230 attaches over one end of sheave pin load cell 402 and includes electronics that connect with sheave pin load cell 402. An optical pattern 406 (e.g., a zebra stripe pattern) is applied to one side of pulley 212(1) facing housing 404, and housing 404 includes optical sensors (see optical sensors 502 of FIG. 5) responsive to optical pattern 406 as pulley 212(1) rotates. For example, optical pattern 406 may have alternating regularly spaced colored areas that each represent an angular segment of pulley 212(1). In another example, optical pattern 406 is encoded such that the optical sensors discern an angle (e.g., an absolute position) of pulley 212(1).

FIG. 5 is a block diagram showing sensor module 230 of FIG. 2 in further example detail. Sensor module 230 includes sheave pin load cell 402 of FIGS. 4A and 4B, optical sensors 502, a communication interface 504, optionally a battery 506, an analog-to-digital converter 507 (ADC 507), and a processor 508 communicatively coupled with memory 510. In certain embodiments, ADC 507 is integral with processor 508. Memory 510 stores firmware 512 implemented as machine-readable instructions that include a force monitor 514, a movement monitor 516, and a communication manager 518 that when executed by processor 508 cause sensor module 230 to implement functionality of sensor module 230 as described herein.

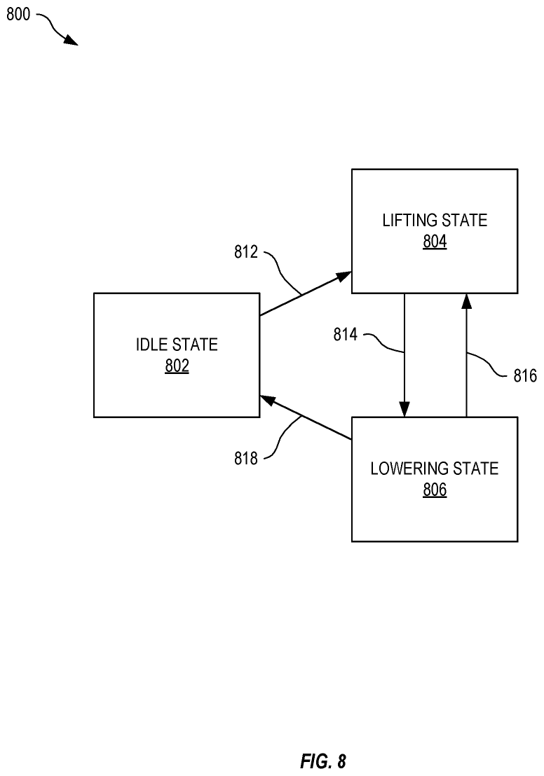

Force monitor 514 causes processor 508 to digitize (e.g., using ADC 507) and process a raw force signal from sheave pin load cell 402 to determine force data 520 that may be stored in memory 510. Movement monitor 516 causes processor 508 to process raw movement data (e.g., optical information) from optical sensors 502 to determine movement data 530 that may be stored in memory 510. Optical sensors 502 detect changes in light intensity as optical pattern 406 moves relative to housing 404 as a result of movement in cable 210 that causes pulley 212(1) to rotate. The detected changes in light intensity indicate changes in angular rotation of pulley 212(1), and may further indicate a direction of rotation. Measured angular rotation of pulley 212(1) may be converted into a distance that cable 210 moves based on a radius of pulley 212(1). In certain embodiments, movement monitor 516 calculates a velocity of cable 210 as it moves and records a starting position and an ending point position for the movement that allows a movement distance for the exercise to be determined. For example, movement monitor may include a state machine (e.g., see FIG. 8) that tracks movement of an exercise being performed by a user on indoor strength fitness equipment 201. Advantageously, sensor module 230 provides information that allows interface module 240, or other connected devices, to track velocity based exercise plans and record a range of motion being made by the user. For example, interface module 240 may monitor the range of motion and indicate (e.g., by emitting a warning tone or displaying a warning message) when indoor strength fitness equipment 201 is not being used safely.

Where calibration data 540 is provided, force monitor 514 corrects force data 520 based on calibration data 540 and movement monitor 516 corrects movement data 530 based on 540. For example, 540 includes one or more correction factors for raw force data determined by ADC 507 from output of sheave pin load cell 402 and one or more correction factors for raw movement data output from optical sensors 502.

Communication manager 518 uses communication interface 504 to communicate force data 520 and movement data 530 to interface module 240 of FIG. 2.

Accordingly, force data 520 and movement data 530 may be used to determine work performed by user 220. For example, work performed is force (force data 520) multiplied by distance (movement data 530).

In certain embodiments, communication interface 504 implements a short-range wireless protocol, such as Bluetooth Low Energy (BLE), ANT+, and/or Wi-Fi that is used by communication manager 518 to wirelessly communicate with interface module 240. In this embodiment, sensor module 230 is self-powered and includes battery 506 to power sheave pin load cell 402, optical sensors 502, communication interface 504, processor 508, and memory 510. Battery 506 may be selected to provide sensor module 230 with a minimum run time of fifty-thousand hours, for example. In other embodiments, communication interface 504 includes circuitry to drive a hard-wired connection between sensor module 230 and interface module 240 and may receive power via the hard-wired connection from interface module 240. In this embodiment, sensor module 230 does not include battery 506 and power received from interface module 240 is used to power sheave pin load cell 402, optical sensors 502, communication interface 504, processor 508, and memory 510.

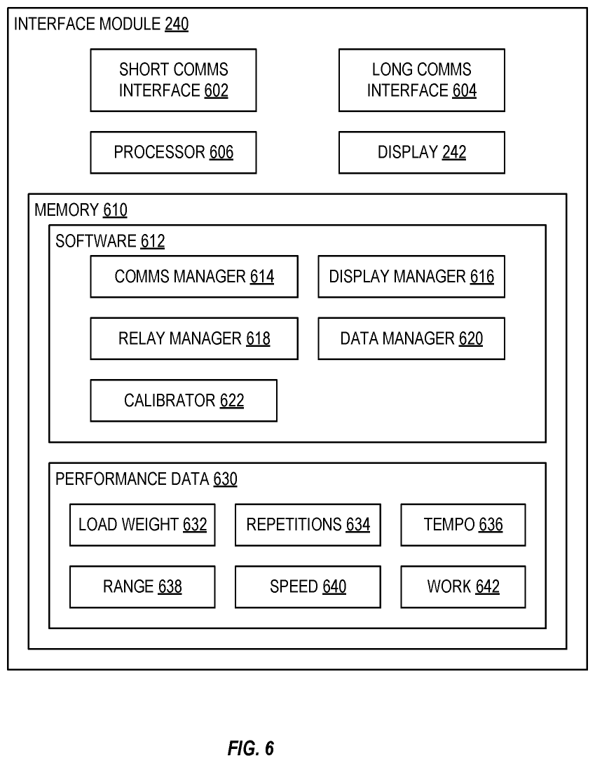

FIG. 6 is a block diagram showing interface module 240 of FIG. 2 in further example detail. Interface module 240 includes a short-range communication interface 602, a long-range communication interface 604, a processor 606, display 242, and memory 610 storing software 612 implemented as machine-readable instructions that, when executed by processor 606, cause interface module 240 to implement a communication manager 614, a display manager 616, a relay manager 618, a data manager 620, and a calibrator 622.

Communication manager 614 controls short-range communication interface 602 to communicate with sensor module 230 (e.g., wireless using BLE and/or ANT+ or wired) to receive force data 520 and movement data 530. Data manager 620 processes force data 520 and movement data 530 to determine performance data 630 for an exercise performed by user 220 on indoor strength fitness equipment 201. For example, data manager 620 processes force data 520 to determine a load weight 632, and processes movement data 530 to determine one or more of repetitions 634, tempo 636, range of motion 638, speed profile 640, and word 642. Data manager 620 may also implement a calibration routine that determines calibration parameters use to process force data 520 and movement data 530 to determine real world values for performance data 630. Performance data 630 may include other metrics determined from force data 520 and movement data 530 without departing from the scope hereof. In certain embodiments, memory 610 includes a buffer for storing multiple sets of performance data 630.

Display manager 616 may control display 242 to output information of performance data 630. For example, display manager 616 may generate one or more charts, tables, and animations corresponding to performance data 630 (e.g., values and charts showing one or more of load weight 632, repetitions 634, tempo 636, range of motion 638 and speed profile 640).

Relay manager 618 may control long-range communication interface 604 to relay performance data 630 to server 260 via Internet 250 for example. Long-range communication interface 604 may implement one or more of Wi-Fi, LORA, and/or cellular protocols.

Calibrator 622 is invoked to calibrate sensor module 230 to indoor strength fitness equipment 201. Sensor module 230 includes calibration data 540 that calibrates forces sensed by sheave pin load cell 402 and/or movement measured by optical sensors 502 based on physical characteristics of indoor strength fitness equipment 201. As described above, sensor module 230 may be retrofitted to existing indoor strength fitness equipment 100, where the type and characteristics of indoor strength fitness equipment 100 is unknown prior to fitting of sensor module 230. Further, sensor module 230 may operate with different types of exercise equipment. Accordingly, once fitted, sensor module 230 may be calibrated to improve the quality of performance data 630 generated therefrom.

In one example of operation, when interface module 240 pairs with sensor module 230, calibrator 622 is invoked to determine whether sensor module 230 has been calibrated. For example, during pairing, sensor module 230 may return a status indicating whether or not it has been calibrated. When the status indicates that calibration data 540 has not been configured within sensor module 230, software 612 may automatically invoke calibrator 622 to prompt a user of interface module 240 to perform a calibration routine. In one example of calibration, calibrator 622 prompts the user to perform at least one predefined operation on the fitness equipment while sensor module 230 captures force data 520 and movement data 530 that is sent to calibrator 622. Calibrator 622 then determines calibration data 540 based on force data 520 and/or movement data 530 and weight and/or movement values of the requested calibration routine and sends calibration data 540 to sensor module 230 where it is stored in memory 510. For example, a calibration routine may instruct the user to select a twenty-pound weight and to move handle 208 a distance of three feet. In certain embodiments, sensor module 230 may be factory calibrated prior to deployment, whereby the calibration process uses known weights. Force monitor 514 and/or movement monitor 516 use calibration data 540 to automatically correct force data 520 and/or movement data 530. Software 612 may also allow the user of interface module 240 to invoke calibrator 622 at other times when needed.

In certain embodiments, interface module 240 may further allow the user to define other characteristics of indoor strength fitness equipment 201. For example, where indoor strength fitness equipment 201 has weight stack 204, the user may also define corresponding weight steps (e.g., five-pound steps, half-kilogram steps, etc.). Accordingly, sensed weights may be restricted to a nearest weight step. For example, interface module 240 may use at least two known weights to determine a raw strain slope to calibrate sensor module 230. Further, interface module 240 may recommend that the user adds or removes weight based on the user's performance during an exercise.

Communication manager 614 may also implement one or more protocol to interface with other fitness equipment. For example, communication manager 614 and/or short-range communication interface 602 may implement an Apple® GymKit protocol, and thereby integrate sensor module 230 and interface module 240 within a fitness environment. In another example, communication manager 614 communicates with a mobile device (e.g., a smartphone, a smart watch, etc.) of a user of indoor strength fitness equipment 201.

### Data Flow

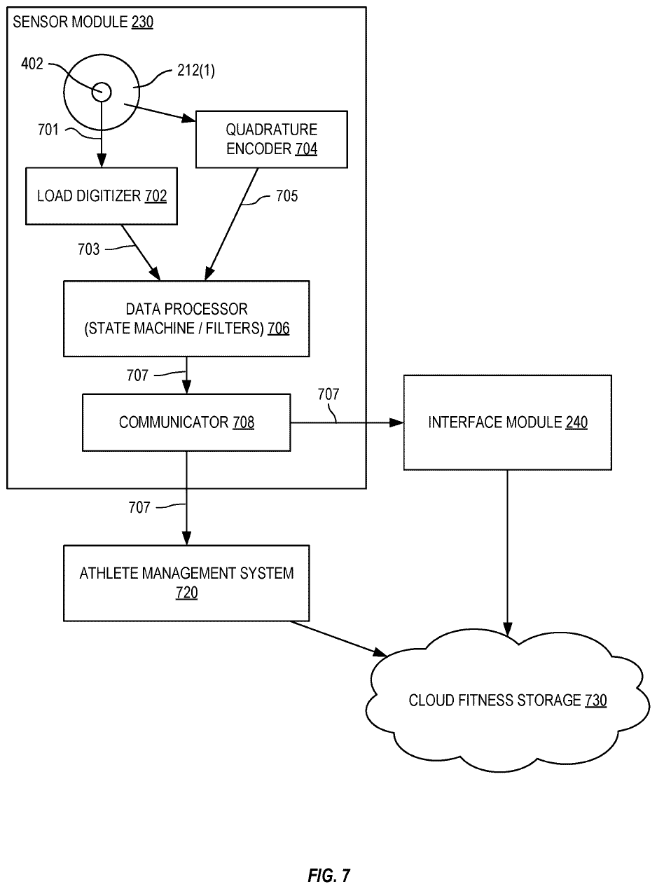

FIG. 7 is a block diagram illustrating example dataflow for improved indoor strength fitness equipment 201 of FIG. 2, in embodiments. Sensor module 230 is represented as a load digitizer 702, a quadrature encoder 704, a data processor 706, and a communicator 708.

Sheave pin load cell 402 outputs a raw force signal 701 representative of a force applied to pulley 212(1) by cable 210. Raw force signal 701 is digitized by load digitizer 702 and input as raw force data 703 to data processor 706. Load digitizer 702 is implemented by ADC 507 of sensor module 230 for example.

Quadrature encoder 704 includes optical sensors 502 that captures raw movement data 705 representative of movement of pulley 212(1) caused by cable 210. Raw movement data 705 defines both a distance (e.g., rotational angle of pulley 212(1)) and direction of movement of pulley 212(1), for example.

Data processor 706 may implement a state machine (see FIG. 8) and/or signal filters for raw force data 703 and/or raw movement data 705 to determine corrected force and movement data 707 that is output via communicator 708. Data processor 706 improves the quality of raw force data 703 and/or raw movement data 705 by removing noise and anomalous values. Data processor 706 also corrects raw force data 703 and/or raw movement data 705, based on calibration data 540, to form corrected force and movement data 707.

Corrected force and movement data 707 is sent to communicator 708 where and transmitted to interface module 240 and/or an athlete management system 720. Athlete management system 720 may be implemented on a local device, such as on a user's watch or smartphone, and may not push the corrected force and movement data 707 to cloud fitness storage 730, unless so configured by the user. In embodiments where interface module 240 is a an electronic device with a display that is mounted with or near indoor strength fitness equipment 201, interface module 240 may be configured to push corrected force and movement data 707 to cloud fitness storage 730. Further, interface module 240 may send other operational data (e.g., use data, wear and predictive maintenance information, and so on) to cloud fitness storage 730 and/or another cloud based server. Interface module 240 and/or athlete management system 720 may store workout information within a cloud fitness storage 730, for example. Athlete management system 720 uses a standard protocol whereby sensor module 230 implements this protocol such that output data 707.

FIG. 8 is a block diagram illustrating one example state machine 800 implemented within sensor module 230 of indoor strength fitness equipment 201 of FIG. 2, in embodiments. State machine 800 includes three states: idle state 802, lifting state 804, and lowering state 806. Idle state 802 is a starting state and occurs when there is no force detected by force monitor 514 and no movement detected by movement monitor 516. State machine 800 makes transition 812 from idle state 802 to lifting state 804 when movement data 530 indicates movement in a direction that corresponds to upward movement of weight stack 204 and force data 520 indicates a weight at least equal to a minimum selectable weight of weight stack 204. State machine 800 makes transition 814 from lifting state 804 to lowering state 806 when movement data 530 indicates movement in a direction that corresponds to downward movement of weight stack 204. State machine 800 makes transition 816 from lowering state 806 to lifting state 804 when movement data 530 indicates movement in a direction that corresponds to upward movement of weight stack 204. State machine 800 makes transition 818 from lowering state 806 to idle state 802 when any of the following occur: (a) no significant motion is detected for a timeout period (e.g., thirty seconds), and (b) force data 520 indicates a weight less than the minimum selectable weight of weight stack 204. In certain embodiments, sensor module 230 may include other types of sensor that detect when a user is no longer using indoor strength fitness equipment 201. In one example, sensor module 230 may include an infrared sensor that detects when the user is no longer using indoor strength fitness equipment 201. In another example, sensor module 230 includes a handgrip sensor that detects when the user is no longer holding handle 208. In these embodiments, state machine 800 may also transition 818 from lowering state 806 to idle state 802 when the user is no longer detected.

At transition 814, (e.g., when state machine 800 transitions from lifting state 804 to lowering state 806), sensor module 230 sends force data 520 and movement data 530 to interface module 240, thereby indicating one ‘rep’ has been performed. Accordingly, interface module 240 updates display 242 to indicate progress of the user in the monitored exercise. At transition 818 (e.g., when state machine 800 transitions from lowering state 806 to idle state 802), sensor module 230 sends an idle message to interface module 240 that may cause interface module 240 to send a workout summary to athlete management system 720 and/or cause interface module 240 to store the workout summary in cloud fitness storage 730.

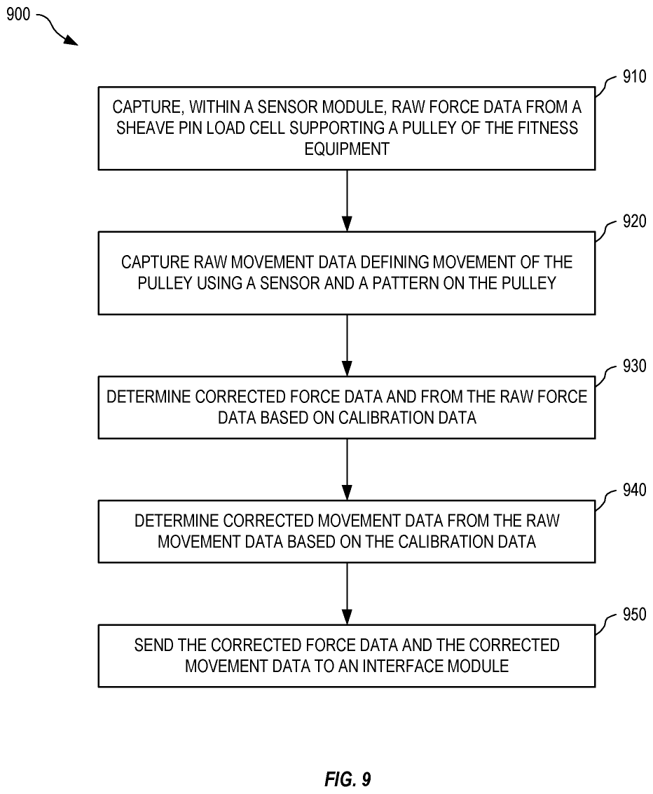

FIG. 9 is a flowchart illustrating one example method 900 for measuring performance of a user performing an exercise on fitness equipment, in embodiments. Method 900 is implemented within connected system 200 of FIG. 2, for example.

In block 910, method 900 captures, within a sensor module, raw force data from a sheave pin load cell supporting a pulley of the indoor strength fitness equipment. In one example of block 910, sensor module 230 captures raw force data 703 from sheave pin load cell 402 that support pulley 212(1) in frame 202 of indoor strength fitness equipment 201.

In block 920, method 900 captures raw movement data defining movement of the pulley using a sensor and a pattern on the pulley. In one example of block 920, optical sensors 502 capture raw movement data 705 indicative of movement of pulley 212(1) based on optical pattern 406.

In block 930, method 900 determines corrected force data from the raw force data based on calibration data. In one example of block 930, force monitor 514 causes processor 508 to correct force data 520 based on calibration data 540.

In block 940, method 900 determines corrected movement data from the raw movement data based on the calibration data. In one example of block 940, movement monitor 516 causes processor 508 to correct movement data 530 based on calibration data 540.

In block 950, method 900 sends the corrected force data and the corrected movement data to an interface module. In one example of block 950, communication manager 518 causes processor 508 to output force data 520 and movement data 530 via communication interface 504.

Method 900 repeats at intervals to measure performance of the user over time.

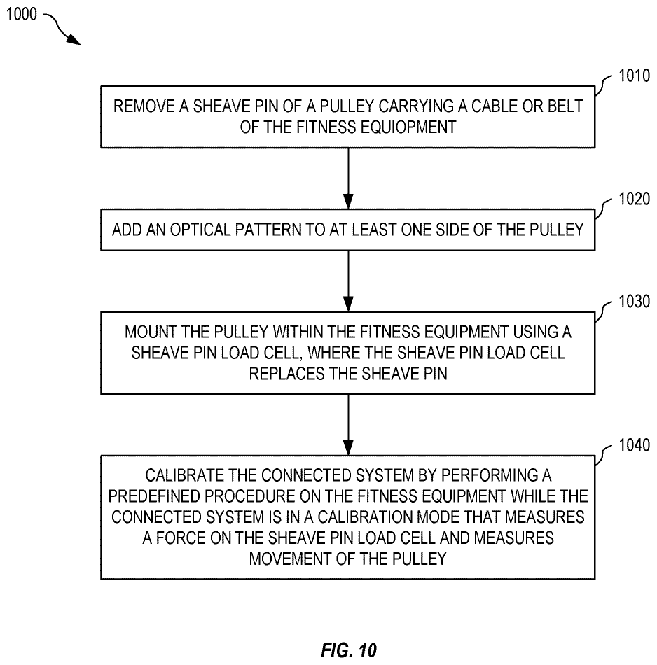

FIG. 10 is a flowchart illustrating one example method 1000 for adding a connected system to fitness equipment. Method 1000 may be performed by a person having basic mechanical skills to remove and replace a pully on the fitness equipment.

In block 1010, method 1000 removes a sheave pin of a pulley carrying a cable or belt of the fitness equipment. In one example of block 1010, a person removes pin 214(1) from indoor strength fitness equipment 201. In block 1020, method 1000 adds an optical pattern to at least one side of the pulley. In one example of block 1020, optical pattern 406 is added to pulley 212(1). In block 1030, method 1000 mounts the pulley within the strength fitness equipment using a sheave pin load cell, where the sheave pin load cell replaces the sheave pin. In one example of block 1030, pulley 212(1) is remounted within indoor strength fitness equipment 201 using sheave pin load cell 402. In block 1040, method 1000 calibrates the connected system by performing a predefined procedure on the strength fitness equipment while the connected system is in a calibration mode that measures a force on the sheave pin load cell and measures movement of the pulley. In one example of block 1040, the user invokes calibrator 622 and is prompted to perform at least one predefined operation on indoor strength fitness equipment 201 while sensor module 230 captures force data 520 and movement data 530 that is sent to calibrator 622. Calibrator 622 then determines calibration data 540 based on force data 520 and/or movement data 530 and weight and/or movement values of the requested calibration routine. Calibrator 622 sends calibration data 540 to sensor module 230 where it is stored in memory 510.

Method 1000 is a relatively simple process that takes approximately ten minutes for any type of fitness equipment that uses a cable or belt with a pully for example.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

### Combination of Features

Features described above as well as those claimed below may be combined in various ways without departing from the scope hereof. The following enumerated examples illustrate some possible, non-limiting combinations:
(A1) A connected system for fitness equipment, includes a sensor module having: a sheave pin load cell sized and shaped to support a pulley of the fitness equipment; a sensor for sensing movement of the pulley as the pulley rotates; a communication interface; a processor; and memory storing machine-readable instructions that when executed by the processor cause the sensor module to: capture raw force data from the sheave pin load cell; capture raw movement data from the sensor; determine corrected force data and from the raw force data based on calibration data; determine corrected movement data from the raw movement data based on the calibration data; and output the corrected force data and the corrected movement data via the communication interface.
(A2) Embodiments of (A1), further including an optical pattern formed on the pulley, wherein the sensor is at least two optical sensors.
(A3) In either of embodiments (A1) or (A2), the fitness equipment including strength fitness equipment.
(A4) Any of the embodiments (A1)-(A3), further including an interface module having: a second communication interface; a long-range wireless communication interface; a second processor; and second memory storing machine-readable instructions that when executed by the second processor causes the interface module to: receive, via the second communication interface, the corrected force data and the corrected movement data; and transmit, via the communication interface using a standard protocol, the corrected force data and the corrected movement data.
(A5) In any of embodiments (A1)-(A4), the communication interface and the second communication interface each including a short-range wireless communication interface, the sensor module further including an independent power source.
(A6) In any of the embodiments (A1)-(A5), the communication interface and the second communication interface being connected by wires, wherein the wires carry power to the sensor module from the interface module.
(A7) In any of the embodiments (A1)-(A6), the interface module further including: a display; and machine-readable instructions stored in the second memory that when executed by the second processor cause the interface module to output information of an exercise performed by a user of the fitness equipment based on the corrected force data and the corrected movement data.
(A8) In any of the embodiments (A1)-(A7), the interface module further including machine-readable instructions stored in the second memory that when executed by the second processor cause the interface module to store the corrected force data and the corrected movement data in cloud fitness storage.
(B1) A method for measuring performance of a user performing an exercise on fitness equipment, including: capturing, within a sensor module, raw force data from a sheave pin load cell supporting a pulley of the fitness equipment; capturing raw movement data defining movement of the pulley using a sensor and a pattern on the pulley; determining corrected force data from the raw force data based on calibration data; determining corrected movement data from the raw movement data based on the calibration data; and sending the corrected force data and the corrected movement data to an interface module.
(B2) Embodiments of (B1), further including filtering at least one of the raw force data and the raw movement data remove noise.
(B3) Either of embodiments (B1) or (B2), further including processing, within the interface module, the corrected force data and the corrected movement data to determine work performed by the user during the exercise.
(B4) Any of the embodiments (B1)-(B3), further including determining, within the interface module and from the corrected movement data, at least one of a repetition count, a tempo, a range of motion, and a speed of motion.
(B5) Any of the embodiments (B1)-(B4), further including outputting at least two of the repetition count, the tempo, the range of motion, the speed of motion, and the work on a display of the interface module.
(B6) In any of the embodiments (B1)-(B5), the sending including detecting, within the sensor module and based on the raw movement data, transitions of a state machine between an idle state, a lifting state, and a lowering state, wherein the sending occurs on a transition to the lowering state.
(B7) Any of the embodiments (B1)-(B6), further including sending an idle message to the interface module to indicate an end of the exercise, the interface module sending a workout summary to an athlete management system and/or to a cloud fitness storage.
(C1) A method for adding a connected system to fitness equipment, including: removing a sheave pin of a pulley carrying a cable or belt of the fitness equipment; adding an optical pattern to at least one side of the pulley; mounting the pulley within the fitness equipment using a sheave pin load cell, where the sheave pin load cell replaces the sheave pin; and calibrating the connected system by performing a predefined procedure on the fitness machine while the connected system is in a calibration mode that measures a force on the sheave pin load cell and measures movement of the pulley.

What is claimed is:

1. A connected system for fitness equipment, comprising a sensor module having:
a sheave pin load cell sized and shaped to support a pulley of the fitness equipment;
a sensor for sensing rotational movement of the pulley as the pulley rotates;
a communication interface;
a processor; and
memory storing machine-readable instructions that when executed by the processor cause the sensor module to:
capture raw force data from the sheave pin load cell;
capture raw movement data from the sensor;
determine corrected force data and from the raw force data based on calibration data;
determine corrected movement data from the raw movement data based on the calibration data; and
output the corrected force data and the corrected movement data via the communication interface.

2. The connected system of claim 1, further comprising an optical pattern formed on the pulley, wherein the sensor is at least two optical sensors.

3. The connected system of claim 1, the fitness equipment comprising strength fitness equipment.

4. The connected system of claim 1, further comprising an interface module having:
a second communication interface;
a long-range wireless communication interface;
a second processor; and
second memory storing machine-readable instructions that when executed by the second processor causes the interface module to:
receive, via the second communication interface, the corrected force data and the corrected movement data; and
transmit, via the communication interface using a standard protocol, the corrected force data and the corrected movement data.

5. The connect system of claim 4, the communication interface and the second communication interface each comprising a short-range wireless communication interface, the sensor module further comprising an independent power source.

6. The connected system of claim 4, the communication interface and the second communication interface being connected by wires, wherein the wires carry power to the sensor module from the interface module.

7. The connected system of claim 4, the interface module further comprising:
a display; and
machine-readable instructions stored in the second memory that when executed by the second processor cause the interface module to output information of an exercise performed by a user of the fitness equipment based on the corrected force data and the corrected movement data.

8. The connected system of claim 4, the interface module further comprising machine-readable instructions stored in the second memory that when executed by the second processor cause the interface module to store the corrected force data and the corrected movement data in cloud fitness storage.

9. A method for measuring performance of a user performing an exercise on fitness equipment, comprising:
capturing, within a sensor module, raw force data from a sheave pin load cell supporting a pulley of the fitness equipment;
capturing raw movement data defining movement of the pulley using a sensor and a pattern on the pulley;
determining corrected force data from the raw force data based on calibration data;
determining corrected movement data from the raw movement data based on the calibration data; and
sending the corrected force data and the corrected movement data to an interface module.

10. The method of claim 9, further comprising filtering at least one of the raw force data and the raw movement data remove noise.

11. The method of claim 9, further comprising processing, within the interface module, the corrected force data and the corrected movement data to determine work performed by the user during the exercise.

12. The method of claim 11, further comprising determining, within the interface module and from the corrected movement data, at least one of a repetition count, a tempo, a range of motion, and a speed of motion.

13. The method of claim 12, further comprising outputting at least two of the repetition count, the tempo, the range of motion, the speed of motion, and the work on a display of the interface module.

14. The method of claim 9, the sending comprising detecting, within the sensor module and based on the raw movement data, transitions of a state machine between an idle state, a lifting state, and a lowering state, wherein the sending occurs on a transition to the lowering state.

15. The method of claim 14, further comprising sending an idle message to the interface module to indicate an end of the exercise, the interface module sending a workout summary to an athlete management system and/or to a cloud fitness storage.

16. A method for adding a connected system to fitness equipment, comprising:
removing a sheave pin of a pulley carrying a cable or belt of the fitness equipment;
adding an optical pattern to at least one side of the pulley;
mounting the pulley within the fitness equipment using a sheave pin load cell, where the sheave pin load cell replaces the sheave pin; and
calibrating the connected system by performing a predefined procedure on the fitness equipment while the connected system is in a calibration mode that measures a force on the sheave pin load cell and measures movement of the pulley.
